SEQUENCE LISTING (2 sequences)

SEQ ID NO: 1 - DNA, 1295 bp, Homo sapien
cgccactttgctggagcattcactaggcgaggcgctccatcggactcactagccgcactcatgaatcggcaccatctgcaggatcactttctggaaatagacaagaagaactgctgtgtgttccgagatgacttcattgtcaaggtgttgccgccggtgttggggctggagtttatcttcgggcttctgggcaatggccttgccctgtggattttctgtttccacctcaagtcctggaaatccagccggattttcctgttcaacctggcagtggctgactttctactgatcatctgcctgcccttcctgatggacaactatgtgaggcgttgggactggaagtttggggacatcccttgccggctgatgctcttcatgttggctatgaaccgccagggcagcatcatcttcctcacggtggtggcggtagacaggtatttccgggtggtccatccccaccacgccctgaacaagatctccaatcggacagcagccatcatctcttgccttctgtggggcatcactattggcctgacagtccacctcctgaagaagaagatgccgatccagaatggcggtgcaaatttgtgcagcagcttcagcatctgccataccttccagtggcacgaagccatgttcctcctggagttcttcctgcccctgggcatcatcctgttctgctcagccagaattatctggagcctgcggcagagacaaatggaccggcatgccaagatcaagagagccatcaccttcatcatggtggtggccatcgtctttgtcatctgcttccttcccagcgtggttgtgcggatccgcatcttctggctcctgcacacttcgggcacgcagaattgtgaagtgtaccgctcggtggacctggcgttctttatcactctcagcttcacctacatgaacagcatgctggaccccgtggtgtactatttttccagcccatcctttcccaacttcttctccactttgatcaaccgctgcctccagaggaagatgacaggtgagccagataataaccgcagcacgagcgtcgagctcacaggggaccccaacaaaaccagaggcgctccagaggcgttaatggccaactccggtgagccatggagcccctcttatctgggcccaacctctccttaaataaccatgccaagaagggacattgtcaccaagaaccagcatctctggagaaacagttgggctgttgcatcgagtaatgtcactggactcggcctaaggtttcctggaacttccagattcagagaatctgatttagggaaact

SEQ ID NO: 2 - protein, 363 aa, Homo sapien [sequence omitted]

(19) United States
(12) Patent Application Publication
(10) Pub. No.: US 2007/0191378 A1
(43) Pub. Date: Aug. 16, 2007

(54) Anthranilic acid derivatives and their use as activators of the hm74a receptor

(75) Inventors: Matthew Campbell, Hertfordshire (GB); Richard Jonathan Hatley, Hertfordshire (GB); Jag Paul Heer, Essex (GB); Andrew McMurtrie Mason, Hertfordshire (GB); Ivan Leo Pinto, Hertfordshire (GB); Shahzad Sharooq Rahman, Essex (GB); Ian Edward David Smith, Hertfordshire (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: SMITHKLINE BEECHAM CORPORATION, Philadelphia, PA (US)

(21) Appl. No.: 10/568,029

(22) PCT Filed: Aug. 13, 2004
(86) PCT No.: PCT/GB04/03516

§ 371 Date: Sep. 15, 2006

(30) Foreign Application Priority Data
Aug. 14, 2003 (GB) 0319126.9

(51) Int. Cl. A61K 31/498; A61K 31/47; A61K 31/195; C07D 217/12; C07D 215/08

(52) U.S. Cl. 514/249; 514/310; 514/311; 514/563; 544/353; 546/146; 546/169; 562/450

(57) ABSTRACT

Therapeutically active anthranilic acid derivatives of Formula (I), processes for the preparation of said derivatives, pharmaceutical formulations containing the compounds and the use of the compounds in therapy, particularly in the treatment of diseases in which under-activation of the HM74A receptor contributes to the disease or in which activation of the receptor will be beneficial, are disclosed.

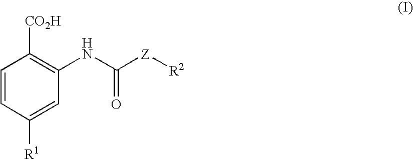

The present invention relates to therapeutically active compounds which are anthranilic acid derivatives, processes for the manufacture of said derivatives, pharmaceutical formulations containing the active compounds and the use of the compounds in therapy, particularly in the treatment of diseases in which under-activation of the HM74A receptor contributes to the disease or in which activation of the receptor will be beneficial.

Dyslipidaemia is a general term used to describe individuals with aberrant lipoprotein profiles. Clinically, the main classes of compounds used for the treatment of patients with dyslipidaemia, and therefore at risk of cardiovascular disease are the statins, fibrates, bile-acid binding resins and nicotinic acid. Nicotinic acid (Niacin, a B vitamin) has been used clinically for over 40 years in patients with various forms of dyslipidaemia. The primary mode of action of nicotinic acid is via inhibition of hormone-sensitive triglyceride lipase (HSL), which results in a lowering of plasma non-esterified fatty acids (NEFA) which in turn alters hepatic fat metabolism to reduce the output of LDL and VLDL (low and very low density lipoprotein). Reduced VLDL levels are thought to lower cholesterol ester transfer protein (CETP) activity to result in increased HDL (high density lipoprotein) levels which may be the cause of the observed cardiovascular benefits. Thus, nicotinic acid produces a very desirable alteration in lipoprotein profiles; reducing levels of VLDL and LDL whilst increasing HDL. Nicotinic acid has also been demonstrated to have disease modifying benefits, reducing the progression and increasing the regression of atherosclerotic lesions and reducing the number of cardiovascular events in several trials.

The observed inhibition of HSL by nicotinic acid treatment is mediated by a decrease in cellular cyclic adenosine monophosphate (cAMP) caused by the G-protein-mediated inhibition of adenylyl cyclase. Recently, the G-protein coupled receptors HM74 and HM74A have been identified as receptors for nicotinic acid (PCT patent application WO02/84298; Wise et. al. J Biol Chem. 2003 278 (11) 9869-9874). The DNA sequence of human HM74A may be found in Genbank; accession number AY148884. Two other papers support this discovery, (Tunaru et. al. Nature Medicine 2003 (3) 352-255 and Soga et. al. Biochem Biophys Res Commun. 2003 303 (1) 364-369), however the nomenclature differs slightly. In the Tunaru paper what they term human HM74 is in fact HM74A and in the Soga paper HM74b is identical to HM74A. Cells transfected to express HM74A and/or HM74 gain the ability to elicit Gₗ G-protein mediated responses following exposure to nicotinic acid. In mice lacking the homologue of HM74A (m-PUMA-G) nicotinic acid fails to reduce plasma NEFA levels.

Certain anthranilic acid derivatives have been synthesised and disclosed in the prior art. For example, U.S. Pat. No. 5,075,313 and Yu, Melvin J. et. al. J. Med. Chem. 1992, vol. 35, 2534-2542 both relate to 3-aryl-4(3H)quinazolinone CCK antagonists useful in treating CNS and gastrointestinal disorders and discloses certain anthranilic acid derivatives as intermediates in their synthesis.

We now present a group of anthranilic acid derivatives which are selective agonists of the nicotinic acid receptor HM74A and are thus of benefit in the treatment, prophylaxis and suppression of diseases in which under-activation of this receptor either contributes to the disease or in which activation of the receptor will be beneficial.

### SUMMARY OF THE INVENTION

The present invention provides therapeutically active anthranilic acid derivatives and the use of these derivatives in therapy, particularly in the treatment of diseases in which under-activation of the HM74A receptor contributes to the disease or in which activation of the receptor will be beneficial, in particular diseases of lipid metabolism including dislipidaemia or hyperlipoproteinaemia such as diabetic dyslipidaemia and mixed dyslipidaemia, heart failure, hypercholesterolaemia, cardiovascular disease including atherosclerosis, arteriosclerosis, and hypertriglyceridaemia. As such, the compounds may also find favour as therapeutics for coronary artery disease, thrombosis, angina, chronic renal failure, peripheral vascular disease and stroke, as well as the cardiovascular indications associated with type II diabetes mellitus, type I diabetes, insulin resistance, hyperlipidaemia, anorexia nervosa, obesity. The compounds may also be of use in the treatment of inflammatory diseases or conditions, as set out further below.

Intermediates, formulations, methods and processes described herein form further aspects of the invention.

### DETAILED DESCRIPTION OF THE INVENTION

According to one aspect of this invention, we provide a compound of Formula (I) 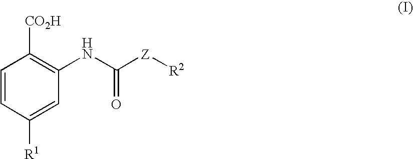
and salts, solvates and physiologically functional derivatives thereof, wherein:

R¹ represents hydrogen, halogen or C₁-C₃alkyl;

R² represents a 9 or 10-member saturated, partially saturated or unsaturated bi-cyclic ring system optionally including from 1 to 3 heteroatoms independently selected from S, O and N;

Z represents a linker unit selected from: —(CH₂)ₙ—; —CH═CH—(CH₂)ₘ—; —(CH₂)ₚNHC(O)—; —(CH₂)ₚNHC(O)NH—; —(CH₂)ₚNHC(O)O—; —(CH₂)ₚSO₂NR³—; —(CH₂)ₚNR³SO₂—; —(CH₂)ₚO— and —O—;

n represents an integer selected from 2, 3 and 4;

m represents an integer selected from 0, 1 and 2;

p represents an integer selected from 1 and 2; and

R³ represents hydrogen or C₁-C₄alkyl;

with the proviso that when R¹ is H, Z is —(CH₂)ₙ— and n=2 or 3, R² is other than indol-3-yl.

The compounds are of use in the treatment of diseases where under-activation of the HM74A receptor contributes to the disease or where activation of the receptor will be beneficial, in particular diseases of lipid metabolism including dislipidaemia or hyperlipoproteinaemia such as diabetic dyslipidaemia and mixed dyslipidaemia, heart failure, hypercholesterolaemia, cardiovascular disease including atherosclerosis, arteriosclerosis, and hypertriglyceridaemia. As such, the compounds may also find favour as therapeutics for coronary artery disease, thrombosis, angina, chronic renal failure, peripheral vascular disease and stroke, as well as the cardiovascular indications associated with type II diabetes mellitus, type I diabetes, insulin resistance, hyperlipidaemia, anorexia nervosa, obesity.

In certain embodiments, R¹ groups are hydrogen, fluorine or methyl (e.g. hydrogen).

In certain particular embodiments, Z represents —(CH₂)ₙ—, —(CH₂)ₚO— or —CH═CH—(CH₂)ₘ—.

In particular embodiments of the invention, n represents 2 or 3 (e.g. 2).

In certain compounds of the invention, m represents 0 or 1.

In particular embodiments, p represents 1.

R³ represents hydrogen or methyl in certain embodiments of the invention.

R² may represent a biaryl, hetero-biaryl, fused aryl-cycloalkyl, fused heteroaryl-cycloalkyl, fused aryl-heterocycle or fused heteroaryl-heterocyclic ring system, as herein defined. In certain embodiments in which R² includes heteroatoms, 1 to 3 heteroatoms are present. The R² ring system may be joined to the Z linker unit via either a ring carbon atom or via a heteroatom, where present.

In certain compounds of the present invention in which the R² unit is a 10-member ring system, this is may be naphthyl or may have either 1 or 2 heteroatoms. Where 2 heteroatoms are present, particular embodiments will have both in the same ring of the fused system. In particular embodiments, the heteroatoms in a 10-member ring system are nitrogen atoms. In certain embodiments, a 10-member R² group is selected from the group consisting of:

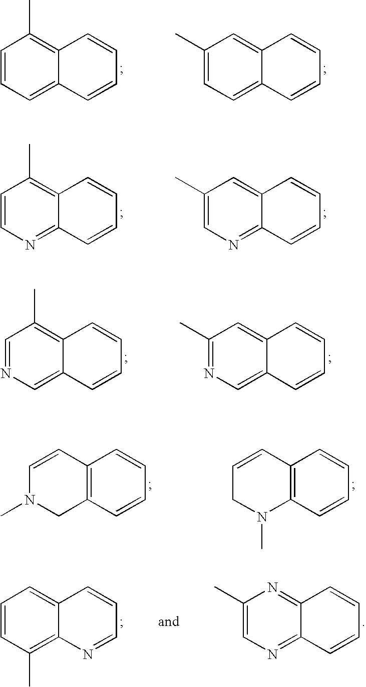

Where the R² unit is a 10-member ring system, this may be unsubstituted. In certain embodiments in which R² is a substituted 10-member ring system, the substituents are selected from C₁-C₂alkyl, (e.g. methyl), —C(O)Me, ═O and C₁-C₃alkoxy (e.g. methoxy).

Where the R² unit is a 9-member ring system, this may be fused aryl-cycloalkyl, for example: 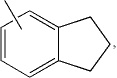
optionally including up to 3 heteroatoms selected from S, O or N, or may be a 9-member fused aryl or heteroaryl system, optionally including up to 3 heteroatoms selected from S, O or N. In particular embodiments in which the R² unit is a 9-member ring system containing heteroatoms, these may be situated in the 5-member ring of the fused system. In particulate embodiments where more than one heteroatom is present, they are both the same such as, for example, a benzimidazole derivative, although heterogeneous heteroaryl systems are also included. In certain embodiments, a 9-member R² group is selected from the group consisting of: 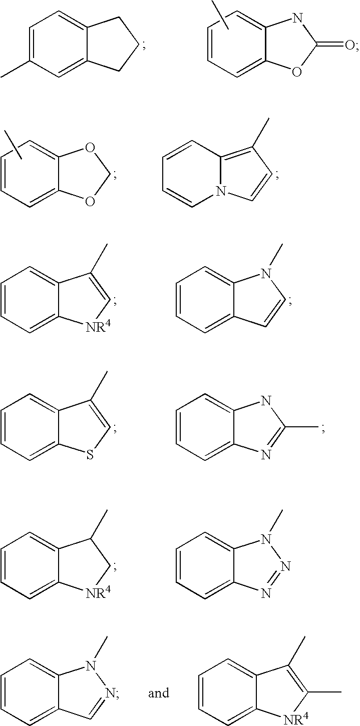
wherein R⁴ represents hydrogen, methyl, CO₂H or CO₂Me.

Where the R² unit is a 9-member ring system, including those depicted above, this may be unsubstituted. In certain embodiments in which R² is a substituted 9-member ring system, the one or more substituents are selected from C₁-C₂alkyl (e.g. methyl); —C(O)Me; ═O; C₁-C₃alkoxy (e.g. methoxy); CO₂H; and CO₂Me.

In certain embodiments:
where the linker unit Z is —(CH₂)ₙ—, n is 2 or 3, more particularly 2;
where Z is —CH═CH—(CH₂)ₘ—, m is 0 or 1, more particularly 0 (in which case Z is —CH═CH—); and
where Z is —(CH₂)ₚO—; —(CH₂)ₚNHC(O)—; —(CH₂)ₚNHC(O)NH—; —(CH₂)ₚNHC(O)O—; —(CH₂)ₚSO₂NR³— or —(CH₂)ₚNR³SO₂—, p is 1.

It is to be understood that the present invention includes any combination of particular embodiments and covers all combinations of particular substituents described hereinabove.

Throughout the present specification and the accompanying claims the words “comprise” and “include” and variations such as “comprises”, “comprising”, “includes” and “including” are to be interpreted inclusively. That is, these words are intended to convey the possible inclusion of other elements or integers not specifically recited, where the context allows.

As used herein, the terms “halogen” or “halo” refer to fluorine, chlorine, bromine and iodine.

As used herein, the term “alkyl” (when used as a group or as part of a group) refers to a straight or branched hydrocarbon chain containing the specified number of carbon atoms. For example, C₁-C₃alkyl means a straight or branched hydrocarbon chain containing at least 1 and at most 3 carbon atoms. Examples of alkyl as used herein include, but are not limited to; methyl (Me), ethyl (Et), n-propyl, i-propyl.

As used herein, the term “alkoxy” (when used as a group or as part of a group) refers to an alkyl ether radical, wherein the term “alkyl” is defined above. Examples of alkoxy as used herein include, but are not limited to; methoxy, ethoxy, n-propoxy, i-propoxy and the like.

As used herein, the term “biaryl” (when used as a group or as part of a group) refers to a group containing two aromatic rings which have two atoms in common. Examples of fused biaryl as used herein include, but are not limited to naphthyl and indyl. Said biaryl groups may be optionally substituted with one or more groups selected from C₁-C₃alkyl, C₁-C₃alkoxy, —C(O)Me, CO₂H, CO₂Me and ═O.

As used herein, the term “hetero-biaryl” (when used as a group or as part of a group) refers to a biaryl group which contains one or more nitrogen, sulphur, or oxygen heteroatoms. Examples of hetero-biaryl as used herein include, but are not limited to; quinoline, isoquinoline, quinoxaline, benzimidazole, indolizine, indole and benzothiophene groups. Said hetero-biaryl groups may be optionally substituted with one or more groups selected from C₁-C₃alkyl, C₁-C₃alkoxy, —C(O)Me, CO₂H, CO₂Me and ═O.

As used herein, the term “fused aryl-cycloalkyl” (when used as a group or as part of a group) refers to a group containing one aromatic ring and one alicyclic ring which have two atoms in common. Examples of fused aryl-cycloalkyl as used herein include, but are not limited to;

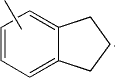

Said fused aryl-cycloalkyl groups may be optionally substituted with one or more groups selected from C₁-C₃alkyl, C₁-C₃alkoxy, —C(O)Me, CO₂H, CO₂Me and ═O.

As used herein, the term “fused heteroaryl-cycloalkyl” (when used as a group or as part of a group) refers to a fused aryl-cycloalkyl group, the aryl ring of which contains one or more nitrogen, sulphur, or oxygen heteroatoms. Said fused heteroaryl-cycloalkyl groups may be optionally substituted with one or more groups selected from C₁-C₃alkyl, C₁-C₃alkoxy, —C(O)Me, CO₂H, CO₂Me and ═O.

As used herein, the term “fused aryl-heterocycle” (when used as a group or as part of a group) refers to a fused aryl-cycloalkyl group, the alicyclic ring of which contains one or more nitrogen, sulphur, or oxygen heteroatoms. Examples of fused aryl-heterocycle as used herein include, but are not limited to; benzodioxolane, indoline. Said fused aryl-heterocycle groups may be optionally substituted with one or more groups selected from C₁-C₃alkyl, C₁-C₃alkoxy, —C(O)Me, CO₂H, CO₂Me and ═O.

As used herein, the term “fused heteroaryl-heterocyclic” (when used as a group or as part of a group) refers to a fused aryl-cycloalkyl group, which contains one or more nitrogen, sulphur, or oxygen heteroatoms either present as an atom shared between the two rings, or one or more heteroatoms being present in each ring. Said fused heteroaryl-heterocyclic groups may be optionally substituted with one or more groups selected from C₁-C₃alkyl, C₁-C₃alkoxy, —C(O)Me, CO₂H, CO₂Me and ═O.

As used herein, the term “physiologically functional derivative” refers to any pharmaceutically acceptable derivative of a compound of the present invention, for example an ester or an amide thereof, and includes any pharmaceutically acceptable salt, ester, or salt of such ester of a compound of formula (I) which, upon administration to a mammal, such as a human, is capable of providing (directly or indirectly) a compound of formula (I) or an active metabolite or residue thereof. It will be appreciated by those skilled in the art that the compounds of formula (I) may be modified to provide physiologically functional derivatives thereof at any of the functional groups in the compounds, and that the compounds of formula (I) may be so modified at more than one position.

As used herein, the term “pharmaceutically acceptable” used in relation to an ingredient (active ingredient or excipient) which may be included in a pharmaceutical formulation for administration to a patient, refers to that ingredient being acceptable in the sense of being compatible with any other ingredients present in the pharmaceutical formulation and not being deleterious to the recipient thereof.

As used herein, the term “solvate” refers to a complex of variable stochiometry formed by a solute (in this invention, a compound of formula (I), a salt thereof or a physiologically functional derivative thereof) and a solvent. Such solvents for the purposes of the present invention may not interfere with the biological activity of the solute. Examples of suitable solvents include water, methanol, ethanol and acetic acid. Preferably the solvent used is a pharmaceutically acceptable solvent. Examples of suitable pharmaceutically acceptable solvents include water, ethanol and acetic acid. Most preferably the solvent used is water, in which case the solvate may be referred to as a hydrate of the solute in question.

It will be appreciated that, for pharmaceutical use, the “salt or solvate” referred to above will be a pharmaceutically acceptable salt or solvate. However, other salts or solvates may find use, for example, in the preparation of a compound of formula (I) or in the preparation of a pharmaceutically acceptable salt or solvate thereof.

Pharmaceutically acceptable salts include those described by Berge, Bighley and Monkhouse, J. Pharm. Sci., 1977, 66, 1-19. Suitable pharmaceutically acceptable salts include acid addition salts formed from the addition of inorganic acids or organic acids, preferably inorganic acids. Examples of suitable acid addition salts include hydrochlorides, hydrobromides, sulphates and acetates. Further representative examples of pharmaceutically acceptable salts include those formed from maleic, fumaric, benzoic, ascorbic, pamoic, succinic, bismethylenesalicylic, methanesulfonic, ethanedisulfonic, propionic, tartaric, salicylic, citric, gluconic, aspartic, stearic, palmitic, itaconic, glycolic, p-aminobenzoic, glutamic, benzenesulfonic, cyclohexylsulfamic, phosphoric and nitric acids. Suitable pharmaceutically acceptable salts also include alkali metal salts formed from the addition of alkali metal bases such as alkali metal hydroxides. An example of a suitable alkali metal salt is a sodium salt.

Compounds of the present invention are of potential therapeutic benefit in the treatment and amelioration of the symptoms of many diseases of lipid metabolism including dislipidaemia or hyperlipoproteinaemia such as diabetic dyslipidaemia and mixed dyslipidaemia, heart failure, hypercholesterolaemia, cardiovascular disease including atherosclerosis, arteriosclerosis, and hypertriglyceridaemia. As such, the compounds may also find favour as therapeutics for coronary artery disease, thrombosis, angina, chronic renal failure, peripheral vascular disease and stroke, as well as the cardiovascular indications associated with type II diabetes mellitus, type I diabetes, insulin resistance, hyperlipidaemia, anorexia nervosa, obesity. The use of a compound of Formula (I) in the treatment of one or more of these diseases is a further aspect of the present invention.

Furthermore, it is also believed that the HM74 and HM74A receptors are involved in inflammation. Inflammation represents a group of vascular, cellular and neurological responses to trauma. Inflammation can be characterized as the movement of inflammatory cells such as monocytes, neutrophils and granulocytes into the tissues.

This is usually associated with reduced endothelial barrier function and oedema into the tissues. Inflammation with regards to disease typically is referred to as chronic inflammation and can last up to a lifetime. Such chronic inflammation may manifest itself through disease symptoms. The aim of anti-inflammatory therapy is therefore to reduce this chronic inflammation and allow for the physiological process of healing and tissue repair to progress.

Thus, a further aspect of the present invention resides in the use of a compound of Formula (I) or a salt, solvate or physiologically functional derivative thereof as defined above in the treatment of inflammatory diseases or conditions of the joint, particularly arthritis (e.g. rheumatoid arthritis, osteoarthritis, prosthetic joint failure), or the gastrointestinal tract (e.g. ulcerative colitis, Crohn's disease, and other inflammatory bowel and gastrointestinal diseases, gastritis and mucosal inflammation resulting from infection, the enteropathy provoked by non-steroidal anti-inflammatory drugs), of the lung (e.g. adult respiratory distress syndrome, asthma, cystic fibrosis, or chronic obstructive pulmonary disease), of the heart (e.g. myocarditis), of nervous tissue (e.g. multiple sclerosis), of the pancreas, (e.g. inflammation associated with diabetes melitus and complications thereof), of the kidney (e.g. glomerulonephritis), of the skin (e.g. dermatitis, psoriasis, eczema, urticaria, burn injury), of the eye (e.g. glaucoma) as well as of transplanted organs (e.g. rejection) and multi-organ diseases (e.g. systemic lupus erythematosis, sepsis) and inflammatory sequelae of viral or bacterial infections and inflammatory conditions associated with atherosclerosis and following hypoxic or ischaemic insults (with or without reperfusion), for example in the brain or in ischaemic heart disease.

In particular, the compounds of Formula (I) are useful in the treatment and prevention of inflammation, and cardiovascular diseases or conditions including atherosclerosis, arteriosclerosis, hypertriglyceridemia, and mixed dyslipidaemia.

Thus, there is also provided the use of a compound of Formula (I) or a pharmaceutically acceptable salt, solvate or physiologically functional derivative thereof, in the manufacture of a medicament for the treatment of disorders of lipid metabolism including dislipidaemia or hyperlipoproteinaemia such as diabetic dyslipidaemia and mixed dyslipidaemia, heart failure, hypercholesterolaemia, cardiovascular disease including atherosclerosis, arteriosclerosis, and hypertriglyceridaemia. The compounds are also provided for use in the treatment of coronary artery disease, thrombosis, angina, chronic renal failure, peripheral vascular disease and stroke, as well as the cardiovascular indications associated with type II diabetes mellitus, type I diabetes, insulin resistance, hyperlipidaemia, anorexia nervosa, obesity.

Nicotinic acid has a significant side effect profile, possibly because it is dosed at high level (gram quantities daily). The most common side effect is an intense cutaneous flushing. The compounds of the present invention preferably exhibit reduced side effects compared to nicotinic acid. HM74A has been identified as a high affinity receptor for nicotinic acid whilst HM74 is a lower affinity receptor. The compounds of the present invention are selective for HM74A by which is meant that they show greater affinity for HM74A than for HM74.

The potential for compounds of formula (I) to activate HM74A may be demonstrated, for example, using the following enzyme and in vitro whole cell assays:

In-Vitro Testing

For transient transfections, HEK293T cells (HEK293 cells stably expressing the SV40 large T-antigen) were maintained in DMEM containing 10% foetal calf serum and 2 mM glutamine. Cells were seeded in 90 mm culture dishes and grown to 60-80% confluence (18-24 h) prior to transfection. Human HM74A (GenBank™ accession number AY148884) was subcloned in to a mammalian expression vector (pcDNA3; Invitrogen) and transfected using Lipofectamine reagent. For transfection, 9 μg of DNA was mixed with 30 μl Lipofectamine in 0.6 ml of Opti-MEM (Life Technologies Inc.) and was incubated at room temperature for 30 min prior to the addition of 1.6 ml of Opti-MEM. Cells were exposed to the Lipofectamine/DNA mixture for 5 h and 6 ml of 20% (v/v) foetal calf serum in DMEM was then added. Cells were harvested 48 h after transfection. Pertussis toxin treatment was carried out by supplementation into media at 50 ngml⁻¹ for 16 h. All transient transfection studies involved co-transfection of receptor together with the G_{l/o} G protein, Gₒ₁α.

For generation of stable cell lines the above method was used to transfect CHO—K1 cells seeded in six well dishes grown to 30% confluence. These cells were maintained in DMEM F-12 HAM media containing 10% foetal calf serum and 2 mM glutamine. 48 h post-transfection the media was supplemented with 400 μg/ml Geneticin (G418, Gibco) for selection of antibiotic resistant cells. Clonal CHO—K1 cell lines stably expressing HM74A were confirmed by [³⁵S]-GTPγS binding measurements, following the addition of nicotinic acid.

P2 membrane preparation—Plasma membrane-containing P2 particulate fractions were prepared from cell pastes frozen at −80° C. after harvest. All procedures were carried out at 4° C. Cell pellets were resuspended in 1 ml of 10 mM Tris-HCl and 0.1 mM EDTA, pH 7.5 (buffer A) and by homogenisation for 20 s with a Ultra Turrax followed by passage (5 times) through a 25-gauge needle. Cell Iysates were centrifuged at 1,000 g for 10 min in a microcentrifuge to pellet the nuclei and unbroken cells and P2 particulate fractions were recovered by microcentrifugation at 16,000 g for 30 min. P2 particulate fractions were resuspended in buffer A and stored at −80° C. until required.

[³⁵S]-GTPγS binding—Assays were performed at room temperature either in 96-well format as described previously (Wieland, T. and Jakobs, K. H. (1994) Methods Enzymol. 237, 3-13) or in an adapted protocol carried out in 384-well format.

96-well format: Briefly, membranes (10 μg per point) were diluted to 0.083 mg/ml in assay buffer (20 mM HEPES, 100 mM NaCl, 10 mM MgCl₂, pH7.4) supplemented with saponin (10 mg/l) and pre-incubated with 10 μM GDP. Various concentrations of nicotinic acid or related molecules were added, followed by [³⁵S]-GTPγS (1170 Ci/mmol, Amersham) at 0.3 nM (total vol. of 100 μl) and binding was allowed to proceed at room temperature for 30 min. Non-specific binding was determined by the inclusion of 0.6 mM GTP. Wheatgerm agglutinin SPA beads (Amersham) (0.5 mg) in 25 μl assay buffer were added and the whole was incubated at room temperature for 30 min with agitation. Plates were centrifuged at 1500 g for 5 min and bound [³⁵S]-GTPγS was determined by scintillation counting on a Wallac 1450 microbeta Trilux scintillation counter.

384-well format: Briefly, the dilution of standard or test compounds were prepared and added to a 384-well plate in a volume of 10 μl. Membranes (HM74A or HM74) were diluted in assay buffer (20 mM HEPES, 100 mM NaCl, 10 mM MgCl₂, pH7.4) supplemented with saponin (60 μg/ml), Leadseeker WGA beads (Amersham; 250 μg/well) and 10 μM GDP, so that the 20 μl volume added to each well contains 5 μg of membranes. [³⁵S]-GTPγS (1170 Ci/mmol, Amersham) was diluted (1:1500) in assay buffer and 20 μl added to each well. Following the addition of the radioligand, the plates were sealed, pulse spun and incubated for 4 hours at room temperature. At the end of the incubation period the plates were read on a Leadseeker machine (VIEWLUX PLUS; Perkin-Elmer) to determine the levels of specific binding.

Compounds according to Formula (I) have been synthesised (see synthetic examples below) and tested in one or more of the assays discussed above. The compounds have an EC50 of 5.0 or greater and an efficacy of 30% or greater.

In-Vivo Testing

HM74A agonists are tested in male Spague-Dawley rats (200-250 grammes) which have been fasted for at least 12 hours prior to the study. The compounds are dosed intravenously (5 ml/kg) or by oral gavage (10 ml/kg). Blood samples (0.3 ml tail vein bleed) are taken pre-dose and at three times post-dose (times ranging from 15 minutes to 8 hours post-dose). Each blood sample is transferred to a heparin tube (Becton Dickinson Microtainer, PST LH) and centrifuged (10,000 g for 5 minutes) to produce a plasma sample. The plasma samples are assayed for levels of non-esterified fatty acids (NEFA) using a commercially available kit (Randox). Inhibition of plasma NEFA levels, relative to pre-dose levels, is used as a surrogate for HM74A agonist activity.

In order to determine whether compounds of the invention exhibit the flushing response associated with nicotinic acid, they are dosed to anaesthetised guinea-pigs. Nicotinic acid is used as positive control. Male Dunkin Hartley guinea pigs (300-800 g) are fasted for 12 hours prior to being anaesthetised with a mixture of Ketamine hydrochloride (Vetalar, 40 mg/kg i.m.), Xylazine (Rompun, 8 mg/kg i.m.) and sodium pentobarbitone (Sagatal, 30 mg/kg i.p.). Following anaesthesia a tracheostomy is performed and the animals are mechanically ventilated with room air (10-12 mL/kg, 60 breaths/min). A jugular vein, and a carotid artery, are cannulated for intravenous administration of test compound and collection of blood respectively. An infra-red temperature probe (Extech Instruments) is placed 3-5 mm from the tip of the left ear. Temperature measurements are recorded every minute from 5 minutes prior to test compound or nicotinic acid and up to 40 minutes post-administration of test compound or nicotinic acid. Data is automatically collected on a Psion computer before being transferred for data analysis within an Excel spreadsheet. Prior to, and at frequent time points after compound administration, blood samples (0.3 ml) are taken via the carotid arterial cannula and transferred to Microtainer (BD) tubes containing lithium heparin. The samples are mixed thoroughly on a blood roller and then stored on ice prior to centrifugation at 1200 g for 5 minutes.

As indicated above, compounds of Formula (I) are useful in human or veterinary medicine, in particular as activators of HM74A, in the management of dyslipidaemia and hyperlipoproteinaemia.

Thus, there is provided as a further aspect of the present invention a compound of formula (I) or a pharmaceutically acceptable salt, solvate or physiologically functional derivative thereof, for use in human or veterinary medicine, particularly in the treatment of disorders of lipid metabolism including dislipidaemia or hyperlipoproteinaemia such as diabetic dyslipidaemia and mixed dyslipidaemia, heart failure, hypercholesterolaemia, cardiovascular disease including atherosclerosis, arteriosclerosis, hypertriglyceridaemia, coronary artery disease, thrombosis, angina, chronic renal failure, peripheral vascular disease and stroke, as well as the cardiovascular indications associated with type II diabetes mellitus, type I diabetes, insulin resistance, hyperlipidaemia, anorexia nervosa, obesity.

It will be appreciated that references herein to treatment extend to prophylaxis, prevention of recurrence and suppression of symptoms as well as the treatment of established conditions.

According to another aspect of the invention, there is provided the use of a compound of formula (Ia)

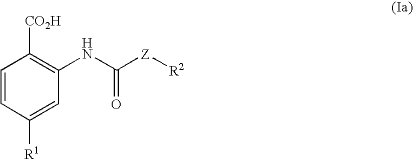

or a pharmaceutically acceptable salt, solvate or physiologically functional derivative thereof, wherein

R¹ represents hydrogen, halogen or C₁-C₃alkyl;

R² represents a 9 or 10-member saturated, partially saturated or unsaturated bi-cyclic ring system optionally including from 1 to 3 heteroatoms independently selected from S, O and N;

Z represents a linker unit selected from: —(CH₂)ₙ—; —CH═CH—(CH₂)ₘ—; —(CH₂)ₚNHC(O)—; —(CH₂)ₚNHC(O)NH—; —(CH₂)ₚNHC(O)O—; —(CH₂)ₚSO₂NR³—; —(CH₂)ₚNR³SO₂—; —(CH₂)ₚO—; and —O—;

n represents an integer selected from 2, 3 and 4;

m represents an integer selected from 0, 1 and 2;

p represents an integer selected from 1 and 2; and

R³ represents hydrogen or C₁-C₄alkyl,

in the manufacture of a medicament for the treatment of disorders of lipid metabolism including dislipidaemia or hyperlipoproteinaemia. In particular, the use is provided of a compound of Formula (Ia) in the manufacture of a medicament for the treatment of diabetic dyslipidaemia, mixed dyslipidaemia, heart failure, hypercholesterolaemia, cardiovascular disease including atherosclerosis, arteriosclerosis, and hypertriglyceridaemia, coronary artery disease, thrombosis, angina, chronic renal failure, peripheral vascular disease and stroke, as well as the cardiovascular indications associated with type II diabetes mellitus, type I diabetes, insulin resistance, hyperlipidaemia, anorexia nervosa, obesity.

In certain embodiments of Formula (Ia), R¹ groups are hydrogen, fluorine or methyl (e.g. hydrogen).

In certain particular embodiments, Z represents —(CH₂)ₙ—, —(CH₂)ₚO— or —CH═CH—(CH₂)ₘ—.

In particular preferred embodiments of the invention, n represents 2 or 3 (e.g. 2).

In certain compounds of the invention, m represents 0 or 1.

In particular embodiments, p represents 1.

R³ represents hydrogen or methyl in certain embodiments of the invention.

R² may represent a biaryl, hetero-biaryl, fused aryl-cycloalkyl, fused heteroaryl-cycloalkyl, fused aryl-heterocycle or fused heteroaryl-heterocyclic ring system, as herein defined. In certain embodiments in which R² includes heteroatoms, 1 to 3 heteroatoms are present. The R² ring system may be joined to the Z linker unit via either a ring carbon atom or via a heteroatom, where present.

In certain compounds of the present invention in which the R² unit is a 10-member ring system, this is may be naphthyl or may have either 1 or 2 heteroatoms. Where 2 heteroatoms are present, particular embodiments will have both in the same ring of the fused system. In particular embodiments, the heteroatoms in a 10-member ring system are nitrogen atoms. In certain embodiments, a 10-member R² group is selected from the group consisting of:

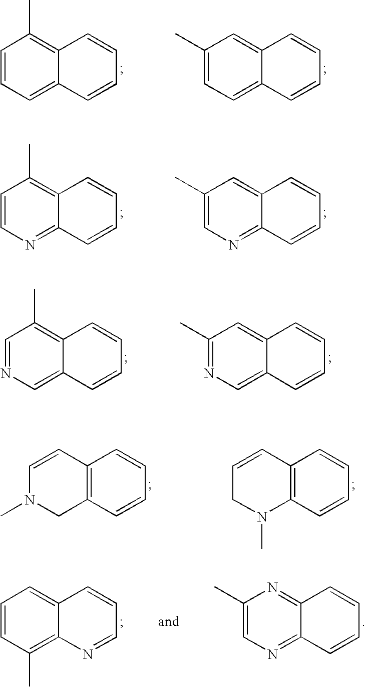

Where the R² unit is a 10-member ring system, this may be unsubstituted. In certain embodiments in which R² is a substituted 10-member ring system, the substituents are selected from C₁-C₂alkyl, (e.g. methyl), —C(O)Me, ═O and C₁-C₃alkoxy (e.g. methoxy).

Where the R² unit is a 9-member ring system, this may be fused aryl-cycloalkyl, for example: 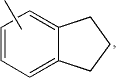
optionally including up to 3 heteroatoms selected from S, O or N, or may be a 9-member fused aryl or heteroaryl system, optionally including up to 3 heteroatoms selected from S, O or N. In particular embodiments in which the R² unit is a 9-member ring system containing heteroatoms, these may be situated in the 5-member ring of the fused system. In particulate embodiments where more than one heteroatom is present, they are both the same such as, for example, a benzimidazole derivative, although heterogeneous heteroaryl systems are also included. In certain embodiments, a 9-member R² group is selected from the group consisting of: 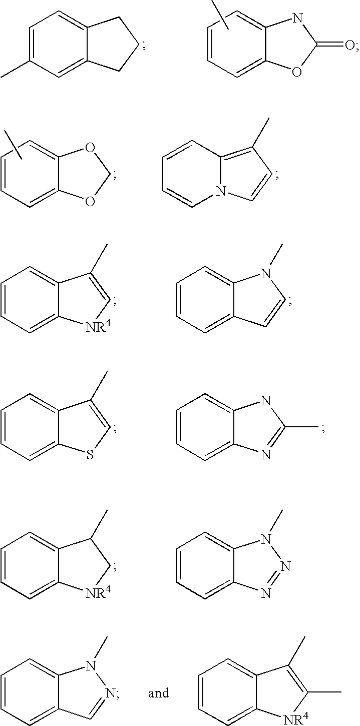
wherein R⁴ represents hydrogen, methyl, CO₂H or CO₂Me.

Where the R² unit is a 9-member ring system, including those depicted above, this may be unsubstituted. In certain embodiments in which R² is a substituted 9-member ring system, the one or more substituents are selected from C₁-C₂alkyl (e.g. methyl); —C(O)Me; ═O; C₁-C₃alkoxy (e.g. methoxy); CO₂H; and CO₂Me.

In certain embodiments:
where the linker unit Z is —CH₂)ₙ—, n is 2 or 3, more particularly 2;
where Z is —CH═CH—(CH₂)ₘ—, m is 0 or 1, more particularly 0 (in which case Z is —CH═CH—); and
where Z is —(CH₂)ₚO—, —(CH₂)ₚNHC(O)—, —(CH₂)ₚNHC(O)NH—, —(CH₂)ₚNHC(O)O—, —(CH₂)ₚSO₂NR³— or —(CH₂)ₚNR³SO₂—, p is 1.

It is to be understood that this aspect of the present invention includes, with respect to the use of compounds of Formula (I) or of Formula (Ia) in the manufacture of a medicament, any combination of particular embodiments and covers all combinations of particular substituents of compounds of Formula (I) or of Formula (Ia) described hereinabove.

Additionally, the present invention provides the use of a compound of formula (Ia) or a physiologically acceptable salt or solvate thereof, in the manufacture of a medicament for the treatment of inflammatory diseases or conditions of the joint, particularly arthritis (e.g. rheumatoid arthritis, osteoarthritis, prosthetic joint failure), or of the gastrointestinal tract (e.g. ulcerative colitis, Crohn's disease, and other inflammatory bowel and gastrointestinal diseases, gastritis and mucosal inflammation resulting from infection, the enteropathy provoked by non-steroidal anti-inflammatory drugs), of the lung (e.g. adult respiratory distress syndrome, asthma, cystic fibrosis, or chronic obstructive pulmonary disease), of the heart (e.g. myocarditis), of nervous tissue (e.g. multiple sclerosis), of the pancreas, (e.g. inflammation associated with diabetes melitus and complications thereof, of the kidney (e.g. glomerulonephritis), of the skin (e.g. dermatitis, psoriasis, eczema, urticaria, burn injury), of the eye (e.g. glaucoma) as well as of transplanted organs (e.g. rejection) and multi-organ diseases (e.g. systemic lupus erythematosis, sepsis) and inflammatory sequelae of viral or bacterial infections and inflammatory conditions associated with atherosclerosis and following hypoxic or ischaemic insults (with or without reperfusion), for example in the brain or in ischaemic heart disease.

In a further or alternative aspect there is provided a method for the treatment of a human or animal subject with a condition where under-activation of the HM74A receptor contributes to the condition or where activation of the receptor will be beneficial, which method comprises administering to said human or animal subject an effective amount of a compound of formula (Ia) or a physiologically acceptable salt or solvate thereof.

Again, is to be understood that this aspect of the present invention includes, with respect to the use of compounds of Formula (I) or of Formula (Ia) in the manufacture of a medicament, any combination of particular embodiments and covers all combinations of particular substituents of compounds of Formula (I) or of Formula (Ia) described hereinabove.

More particularly, the present invention provides a method for the treatment of disorders of lipid metabolism including dislipidaemia or hyperlipoproteinaemia such as diabetic dyslipidaemia and mixed dyslipidaemia, heart failure, hypercholesterolaemia, cardiovascular disease including atherosclerosis, arteriosclerosis, and hypertriglyceridaemia, type II diabetes mellitus, type I diabetes, insulin resistance, hyperlipidaemia, anorexia nervosa, obesity, which method comprises administering to said human or animal subject an effective amount of a compound of formula (Ia) or a physiologically acceptable salt or solvate thereof. As such, these compounds may also find favour in methods for the treatment of coronary artery disease, thrombosis, angina, chronic renal failure, peripheral vascular disease and stroke, which methods comprise administering to said human or animal subject an effective amount of a compound of formula (Ia).

The amount of a HM74A modulator which is required to achieve the desired biological effect will, of course, depend on a number of factors, for example, the mode of administration and the precise clinical condition of the recipient. In general, the daily dose will be in the range of 0.1 mg-1 g/kg, typically 0.1-100 mg/kg. An intravenous dose may, for example, be in the range of 0.01 mg to 0.1 g/kg, typically 0.01 mg to 10 mg/kg, which may conveniently be administered as an infusion of from 0.1 μg to 1 mg, per minute. Infusion fluids suitable for this purpose may contain, for example, from 0.01 μg to 0.1 mg, per millilitre. Unit doses may contain, for example, from 0.01 μg to 1 g of a HM74A modulator. Thus ampoules for injection may contain, for example, from 0.01 μg to 0.1 g and orally administrable unit dose formulations, such as tablets or capsules, may contain, for example, from 0.1 mg to 1 g. No toxicological effects are indicated/expected when a compound of the invention is administered in the above mentioned dosage range.

A compound of the present invention may be employed as the compound per se in the treatment of a disease where under-activation of the HM74A receptor contributes to the disease or where activation of the receptor will be beneficial, but is preferably presented with an acceptable carrier in the form of a pharmaceutical formulation. The carrier must, of course, be acceptable in the sense of being compatible with the other ingredients of the formulation and must not be deleterious to the recipient. The carrier may be a solid or a liquid, or both, and is preferably formulated with the HM74A modulator as a unit-dose formulation, for example, a tablet, which may contain from 0.05% to 95% by weight of the HM74A modulator.

The formulations include those suitable for oral, rectal, topical, buccal (e.g. sub-lingual) and parenteral (e.g. subcutaneous, intramuscular, intradermal or intravenous) administration.

There is also provided according to the invention a process for preparation of such a pharmaceutical composition which comprises mixing the ingredients.

Formulations suitable for oral administration may be presented in discrete units, such as capsules, cachets, lozenges or tablets, each containing a predetermined amount of a HM74A modulator; as a powder or granules; as a solution or a suspension in an aqueous or non-aqueous liquid; or as an oil-in-water or water-in-oil emulsion. In general, the formulations are prepared by uniformly and intimately admixing the active HM74A modulator with a liquid or finely divided solid carrier, or both, and then, if necessary, shaping the product. For example, a tablet may be prepared by compressing or molding a powder or granules of the HM74A modulator optionally with one or more accessory ingredients. Compressed tablets may be prepared by compressing, in a suitable machine, the compound in a free-flowing form, such as a powder or granules optionally mixed with a binder, lubricant, inert diluent and/or surface active/dispersing agent(s). Molded tablets may be made by molding, in a suitable machine, the powdered compound moistened with an inert liquid diluent.

Tablets and capsules for oral administration may contain conventional excipients such as binding agents, for example syrup, acacia, gelatin, sorbitol, tragacanth, mucilage of starch or polyvinyl pyrrolidone; fillers, for example, lactose, microcrystalline cellulose, sugar, maize-starch, calcium phosphate or sorbitol; lubricants, for example, magnesium stearate, stearic acid, talc, polyethylene glycol or silica; disintegrants, for example, potato starch, croscarmellose sodium or sodium starch glycollate; or wetting agents such as sodium lauryl sulphate. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, for example, sorbitol syrup, methyl cellulose, glucose/sugar syrup, gelatin, hydroxymethyl cellulose, carboxymethyl cellulose, aluminium stearate gel or hydrogenated edible fats; emulsifying agents, for example, lecithin, sorbitan mono-oleate or acacia; non-aqueous vehicles (which may include edible oils), for example almond oil, fractionated coconut oil, oily esters, propylene glycol or ethyl alcohol; or preservatives, for example, methyl or propyl p-hydroxybenzoates or sorbic acid. The preparations may also contain buffer salts, flavoring, coloring and/or sweetening agents (e.g. mannitol) as appropriate.

Formulations suitable for buccal (sub-lingual) administration include lozenges comprising a HM74A modulator in a flavored base, usually sucrose and acacia or tragacanth, and pastilles comprising the HM74A modulator in an inert base such as gelatin and glycerin or sucrose and acacia.

Formulations of the present invention suitable for parenteral administration conveniently comprise sterile aqueous preparations of an HM74A modulator, preferably isotonic with the blood of the intended recipient. These preparations are preferably administered intravenously, although administration may also be effected by means of subcutaneous, intramuscular, or intradermal injection. Such preparations may conveniently be prepared by admixing the HM74A modulator with water and rendering the resulting solution sterile and isotonic with the blood. Injectable compositions according to the invention will generally contain from 0.1 to 5% w/w of the HM74A modulator.

Thus, formulations of the present invention suitable for parenteral administration comprising a compound according to the invention may be formulated for parenteral administration by bolus injection or continuous infusion and may be presented in unit dose form, for instance as ampoules, vials, small volume infusions or pre-filled syringes, or in multi-dose containers with an added preservative. The compositions may take such forms as solutions, suspensions, or emulsions in aqueous or non-aqueous vehicles, and may contain formulatory agents such as anti-oxidants, buffers, antimicrobial agents and/or toxicity adjusting agents. Alternatively, the active ingredient may be in powder form for constitution with a suitable vehicle, e.g. sterile, pyrogen-free water, before use. The dry solid presentation may be prepared by filling a sterile powder aseptically into individual sterile containers or by filling a sterile solution aseptically into each container and freeze-drying.

Formulations suitable for rectal administration are preferably presented as unit-dose suppositories. These may be prepared by admixing a HM74A modulator with one or more conventional solid carriers, for example, cocoa butter or glycerides and then shaping the resulting mixture.

Formulations suitable for topical application to the skin preferably take the form of an ointment, cream, lotion, paste, gel, spray, aerosol, or oil. Carriers which may be used include vaseline, lanolin, polyethylene glycols, alcohols, and combinations of two or more thereof. The HM74A modulator is generally present at a concentration of from 0.1 to 15% w/w of the composition, for example, from 0.5 to 2%.

By topical administration as used herein, we include administration by insufflation and inhalation. Examples of various types of preparation for topical administration include ointments, creams, lotions, powders, pessaries, sprays, aerosols, capsules or cartridges for use in an inhaler or insufflator or drops (e.g. eye or nose drops).

Ointments and creams may, for example, be formulated with an aqueous or oily base with the addition of suitable thickening and/or gelling agents and/or solvents. Such bases may thus, for example, include water and/or an oil such as liquid paraffin or a vegetable oil such as arachis oil or castor oil or a solvent such as a polyethylene glycol. Thickening agents which may be used include soft paraffin, aluminium stearate, cetostearyl alcohol, polyethylene glycols, microcrystalline wax and beeswax.

Lotions may be formulated with an aqueous or oily base and will in general also contain one or more emulsifying agents, stabilising agents, dispersing agents, suspending agents or thickening agents.

Powders for external application may be formed with the aid of any suitable powder base, for example, talc, lactose or starch. Drops may be formulated with an aqueous or non-aqueous base also comprising one or more dispersing agents, solubilizing agents or suspending agents.

Spray compositions may be formulated, for example, as aqueous solutions or suspensions or as aerosols delivered from pressurized packs, with the use of a suitable propellant, e.g. dichlorodifluoromethane, trichlorofluoromethane, dichlorotetrafluoroethane, 1,1,1,2,3,3,3-heptafluoropropane, 1,1,1,2-tetrafluorethane, carbon dioxide or other suitable gas.

Capsules and cartridges for use in an inhaler or insufflator, of for example gelatin, may be formulated containing a powder mix of a compound of the invention and a suitable powder base such as lactose or starch.

The pharmaceutical compositions according to the invention may also be used in combination with other therapeutic agents, for example in combination with other classes of dyslipidaemic drugs (e.g. statins, fibrates, bile-acid binding resins or nicotinic acid).

The compounds of the instant invention may be used in combination with one or more other therapeutic agents for example in combination with other classes of dyslipidaemic drugs e.g. 3-hydroxy-3-methylglutaryl-coenzyme A reductase inhibitors (statins) or fibrates or bile acid binding resins or nicotinic acid. The invention thus provides, in a further aspect, the use of such a combination in the treatment of diseases where under-activation of the HM74A receptor contributes to the disease or where activation of the receptor will be beneficial and the use of a compound of formula (I) or a pharmaceutically acceptable salt, solvate or physiologically functional derivative thereof in the manufacture of a medicament for the combination therapy of disorders of lipid metabolism including dislipidaemia or hyperlipoproteinaemia such as diabetic dyslipidaemia and mixed dyslipidaemia, heart failure, hypercholesterolaemia, cardiovascular disease including atherosclerosis, arteriosclerosis, and hypertriglyceridaemia, type II diabetes mellitus, type I diabetes, insulin resistance, hyperlipidaemia, anorexia nervosa or obesity.

When the compounds of the present invention are used in combination with other therapeutic agents, the compounds may be administered either sequentially or simultaneously by any convenient route.

The combinations referred to above may conveniently be presented for use in the form of a pharmaceutical formulation and thus pharmaceutical formulations comprising a combination as defined above optimally together with a pharmaceutically acceptable carrier or excipient comprise a further aspect of the invention. The individual components of such combinations may be administered either sequentially or simultaneously in separate or combined pharmaceutical formulations.

When combined in the same formulation it will be appreciated that the two components must be stable and compatible with each other and the other components of the formulation and may be formulated for administration. When formulated separately they may be provided in any convenient formulation, conveniently in such a manner as are known for such compounds in the art.

When in combination with a second therapeutic agent active against the same disease, the dose of each component may differ from that when the compound is used alone. Appropriate doses will be readily appreciated by those skilled in the art.

The invention thus provides, in a further aspect, a combination comprising a compound of formula (I) or a physiologically acceptable salt or solvate thereof together with another therapeutically active agent.

The combination referred to above may conveniently be presented for use in the form of a pharmaceutical formulation and thus pharmaceutical formulations comprising a combination as defined above together with a pharmaceutically acceptable carrier thereof represent a further aspect of the invention.

The compounds of the formula (I) have useful duration of action.

The compounds of formula (I) and salts and solvates thereof may be prepared by the methodology described hereinafter, constituting a further aspect of this invention.

Abbreviations
THF Tetrahydrofuran
TFA Trifluoroacetic Acid
DMSO Dimethylsulphoxide
HBTU O-Benzotriazol-1-yl-N,N,N′,N′-tetramethyluronium hexafluorophosphate
CDI Carbonyl diimidazole
PyHOTs Pyridinium tosylate

Method A

A process for preparing compounds of the present invention is set out in scheme (a): 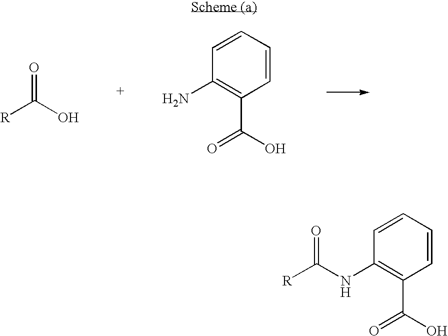
wherein R represents -Z-R² as defined above.

Accordingly, a process according to the invention for preparing a compound of formula (I) comprises:
(i) formation of an amide between the amine group of anthranilic acid (2-amino-bezoic acid) and an activated acyl transfer reagent derived from a carboxylic acid;
(ii) where desired or necessary converting a resultant free acid or base compound of formula (I) into a physiologically acceptable salt form or vice versa or converting one salt form into another physiologically acceptable salt form.

A particular example of a process according to Method A is set out in scheme (a-i): 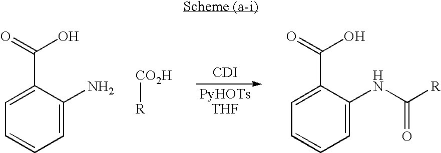
which comprises amide coupling of anthranilic acid with a carboxylic acid using CDI.

Method B

A process for preparing compounds of Formula (I) or Formula (Ia) as defined above in which Z represents —(CH₂)ₚO— and p is 1, is set out in scheme (b): 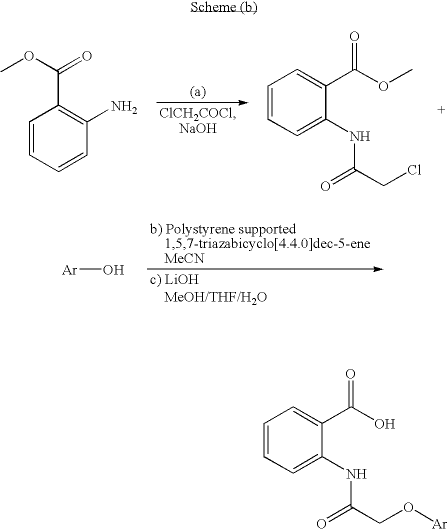
wherein Ar represents R² as defined above
a) amide bond formation by acetylation of an ester of anthranilic acid (O-protected anthranilic acid);
b) Alkylation of aromatic alcohol with resulting methyl 2-[(chloroacetyl)amino]benzoate
c) Hydrolysis of methyl ester using lithium hydroxide (deprotection).

Accordingly, a process according to the invention for preparing a compound of formula (I) comprises:
(i) alkylation of an aromatic alcohol with methyl 2-[(chloroacetyl)amino]benzoate
(ii) hydrolysis of methyl ester using lithium hydroxide
(iii) where desired or necessary converting a resultant free acid or base compound of formula (I) into a physiologically acceptable salt form or vice versa or converting one salt form into another physiologically acceptable salt form.

The following non-limiting examples illustrate the present invention:

### SYNTHETIC EXAMPLES

### A. Example Compounds Synthesised Using Method A

### Example 1

### 2-(3-Naphthalen-1-yl-propanoylamino)-benzoic acid

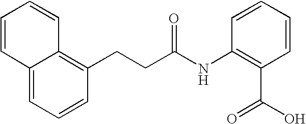

3-Naphthalen-1-yl-propionic acid (20 mgs, 0.1 mmol) was dissolved in acetonitrile (1 ml). HBTU (38 mgs, 0.1 mmol) was added and the mixture stirred for 30 minutes. Then, 2-amino-benzoic acid (14 mgs, 0.1 mmol) was added followed by triethylamine (0.1 ml). The reaction mixture was stirred at room temperature for 20 hours before the addition of water (0.1 ml) and evaporation under reduced pressure. The title compound was isolated using preparative HPLC. δ_{H} (400 MHz, DMSO-d6) 2.81 (2H, t), 3.43 (2H, t), 7.16 (1H, t), 7.43 (2H, d), 7.52-7.58 (3H, m), 7.75-7.82 (1H, m), 7.02-7.98 (2H, m), 8.14 (1H, d), 8.48 (1H, d), 11.24 (1H, br.s), 13.45 (1H, br.s); m/z 318.5 [M−H⁺].

HPLC conditions used for the purification: 8 minute run time. Solvents: 0.1% TFA in MeCN and 0.1% TFA in water. MeCN increased from 10% to 95% linearly over 5 minutes. Held at 95% for 1 min. Decreased to 10% linearly over 30 seconds. Equilibrated at 10% for 1.5 minutes before next injection.

Examples 2 to 23 were prepared as set out in Example 1.

| Example | Structure | LCMS[M − H⁺] |
|---|---|---|
| 2 | 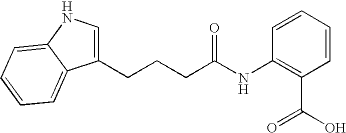 | 321.21 |
| 3 | 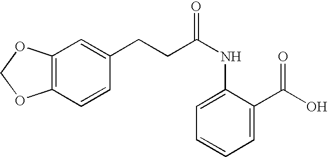 | 312.16 |
| 4 | 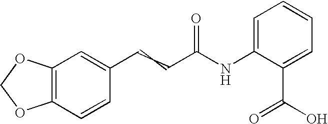 | 310.27 |
| 5 | 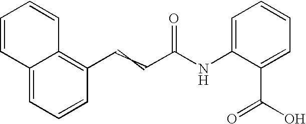 | 319.19[M + H⁺] |
| 6 | 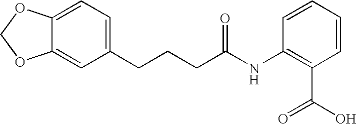 | 325.68 |
| 7 | 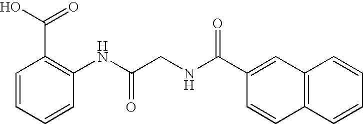 | 347.33 |
| 8 | 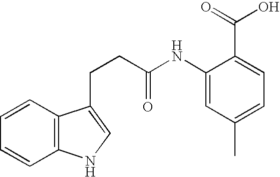 | 321.21 |
| 9 | 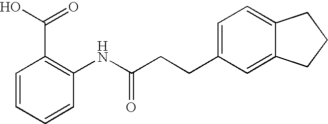 | 308.37 |
| 10 | 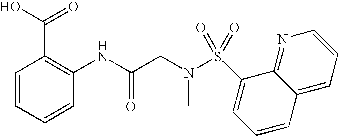 | 398.14 |
| 11 | 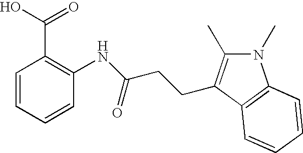 | 335.10 |
| 12 | 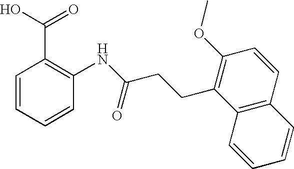 | 348.07 |
| 13 | 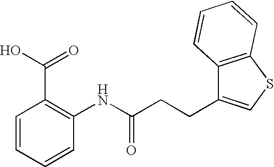 | 324.12 |
| 14 | 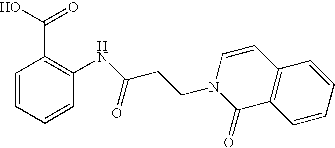 | 335.32 |
| 15 | 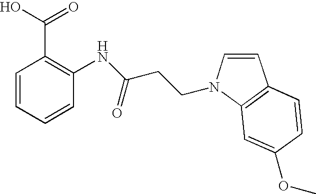 | 339.29[M + H⁺] |
| 16 | 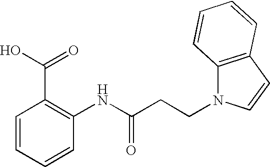 | 309.28[M + H⁺] |
| 17 | 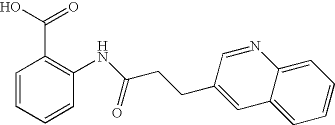 | 319.36 |
| 16 | 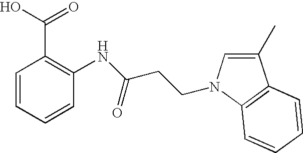 | 323.15[M + H⁺] |
| 19 | 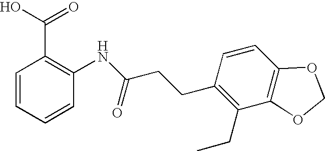 | 339.83 |
| 20 | 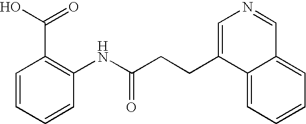 | 319.22 |
| 21 | 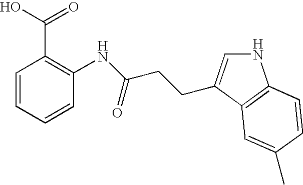 | 321.69 |
| 22 | 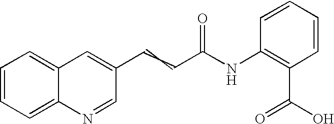 | 317.23 |
| 23 | 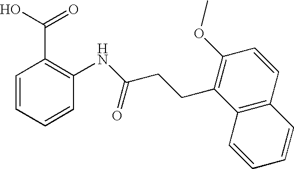 | 348.23 |

### Example 2

δ_{H} (400 MHz, DMSO-d6) 2.00 (2H, m), 2.45 (2H, t), 2.75 (2H, t), 6.96 (1H, t), 7.05 (1H, t), 7.11-7.15 (2H, m), 7.32 (2H, d), 7.51-7.57 (2H, m), 7.96 (1H, d), 8.50 (1H, d), 10.75 (1H, br.s), 11.20 (1H, br.s); m/z 321.21 [M−H⁺].

### Example 4

δ_{H} (400 MHz, DMSO-d6) 6.09 (2H, s), 6.75 (1H, d), 6.96 (1H, d), 7.16-7.22 (2H, m), 7.44 (1H, s), 7.58 (1H, t), 8.00 (1H, d), 8.61 (1H, d), 11.34 (1H, br.s), 13.35 (1H, br.s); m/z 310.27 [M−H⁺].

### Example 5

δ_{H} (400 MHz, DMSO-d6) 7.08 (1H, d), 7.23 (1H, t), 7.66 (1H, t), 7.82 (1H, t), 7.94 (1H, t), 8.03 (1H, d), 8.24 (1H, d), 8.29 (1H, s), 8.31 (1H, d), 8.62 (1H, d), 8.96 (1H, s), 9.40 (1H, s), 11.45 (1H, br.s); m/z 319.19 [M+H⁺].

### Example 6

δ_{H} (400 MHz, DMSO-d6) 1.88 (2H, m), 2.37 (2H, t), 2.56 (2H, t), 5.95 (2H, s), 6.66 (1H, d), 6.81 (2H, m), 7.13 (1H, t), 7.57 (1H, t), 7.96 (1H, d), 8.47 (1H, d), 11.10 (1H, s), 13.74 (1H, br.s); m/z 325.68 [M−H⁺].

### Example 7

δ_{H} (400 MHz, DMSO-d6) 3.98 (2H, d), 7.60 (3H, m), 7.92-8.03 (6H, m), 8.50 (1H, s), 8.97 (1H, t); m/z 347.33 [M−H⁺].

### Example 10

δ_{H} (400 MHz, DMSO-d6) 2.97 (3H, s), 4.33 (2H, s), 7.16 (1H, t), 7.60 (1H, t), 7.70 (1H, m), 7.79 (1H, t), 8.01(1H, d), 8.34 (1H, d), 8.43 (1H, d), 8.55 (2H, t), 9.05 (1H, m); m/z 398.14 [M−H⁺].

### Example 11

δ_{H} (400 MHz, DMSO-d6) 2.34 (3H, s), 2.60 (2H, t), 3.03 (2H, t), 3.62 (3H, s), 6.94 (1H, t), 7.04 (1H, t), 7.10(1H, t), 7.32 (1H, d), 7.48 (1H, d), 7.57 (1H, t), 7.95 (1H, d), 8.50 (1H, d), 11.15 (1H, br.s), 13.41 (1H, br.s); m/z 335.10 [M−H⁺].

### Example 12

δ_{H} (400 MHz, DMSO-d6) 2.59 (2H, t), 3.38 (2H, t), 3.91 (3H, s), 7.15 (1H, t), 7.35 (1H, t), 7.43 (1H, d), 7.50 (1H, t), 7.59 (1H, t), 7.85 (2H, m), 7.97 (1H, d), 8.02 (1H, d), 8.50 (1H, d), 11.20 (1H, br.s), 13.45 (1H, br.s); m/z 348.07 [M−H⁺].

### Example 13

δ_{H} (400 MHz, DMSO-d6) 2.86 (2H, t), 3.19 (2H, t), 7.14 (1H, t), 7.36-7.44 (2H, m), 7.49 (1H, s), 7.58 (1H, t), 7.87 (1H, d), 7.97 (2H, d), 8.48 (1H, d), 11.25 (1H, br.s), 13.55 (1H, br.s); m/z 324.12 [M−H⁺].

### Example 14

δ_{H} (400 MHz, DMSO-d6) 2.90 (2H, t), 4.27 (2H, t), 6.60 (1H, d), 7.15 (1H, t), 7.36-7.44 (2H, m), 7.48-7.52 (2H, m), 7.68 (1H, t), 7.95 (1H, d), 8.22 (1H, d), 8.41 (1H, d), 11.08 (1H, br.s); m/z 335.32 [M−H⁺].

### Example 15

δ_{H} (400 MHz, DMSO-d6) 2.91 (2H, t), 3.79 (3H, s), 4.46 (2H, t), 6.31 (1H, d), 6.65 (1H, dd), 7.07 (1H, d), 7.15 (1H, t), 7.22 (1H, d), 7.37 (1H, d), 7.58 (1H, t), 7.94 (1H, d), 8.43 (1H, d), 11.08 (1H, br.s), 13.53 (1H,br.s); m/z 339.29 [M+H⁺].

### Example 16

δ_{H} (250 MHz, DMSO-d6) 2.93 (2H, t), 4.51 (2H, t), 6.41 (1H, d), 7.01 (1H, t), 7.10-7.17 (2H, m), 7.38 (1H, d), 7.54 (3H, m), 7.94 (1H, dd), 8.40 (1H, d), 11.05 (1H, br.s), 13.52 (1H, br.s); m/z 309.28 [M+H⁺].

### Example 18

δ_{H} (400 MHz, DMSO-d6) 2.21 (3H, s), 2.88 (2H, t), 4.43 (2H, t), 6.99 (1H, t), 7.12 (3H, m), 7.45 (2H, m), 7.56 (1H, t), 7.94 (1H, dd), 8.40 (1H, d), 11.19 (1H, br.s); m/z 323.15 [M+H⁺].

### Example 19

δ_{H} (400 MHz, DMSO-d6) 1.13 (3H, t), 2.60 (4H, m), 2.90 (2H, t), 5.95 (2H, s), 6.68 (2H, m), 7.13 (1H, t), 7.58 (1H, t), 7.97 (1H, d), 8.48 (1H, d), 11.12 (1H, br.s); m/z 339.83 [M−H⁺].

### Example 21

δ_{H} (400 MHz, DMSO-d6) 2.36 (3H, s), 2.74 (2H, t), 3.03 (2H, t), 6.89 (1H, d), 7.08 (1H, s), 7.15 (1H, t), 7.20 (1H, d), 7.31 (1H, s), 7.59 (1H, t), 7.97 (1H, d), 8.51 (1H, d), 10.61 (1H, br.s), 11.24 (1H, br.s); m/z 321.69 [M−H⁺].

Acid intermediates for preparation of examples 2-23 are known compounds, either commercially available or synthesised by reported procedures with the exception of:

### Acid for Example 10

### [Methyl-(quinoline-8-sulfonyl)-amino]-acetic acid:

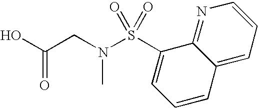

[Methyl-(quinoline-8-sulfonyl)-amino]-acetic acid was prepared from commercially available (quinoline-8-sulfonylamino)-acetic acid by methylation using excess methyl iodide in DMF and subsequent hydrolysis of the methyl ester using a methanolic solution of 2N sodium hydroxide (1:1 mixture).

### Acid for Example 21

### 3-Isoquinolin-4-yl-propionic acid

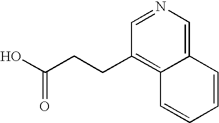

3-Isoquinolin-4-yl-propionic acid prepared from the known compound 3-Isoquinolin-4-yl-propionic acid tert-butyl ester (Jonczyk et al, J. Chem. Res. Synop., 1998, (5), 262-3) by deprotection under standard conditions (20% TFA in DCM for 4 hours).

### Example 24

### 2-{[3-(2-naphthalenyl)propanoyl]amino}benzoic acid

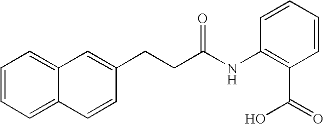

3-(2-naphthalenyl)propanoic acid (150 mg, 0.75 mmol, 1equiv) and 1,1-carbonyldiimidazole (146 mg, 0.9 mmol, 1.2 equiv) were stirred vigorously in anhydrous THF (4 ml) under nitrogen. After 1 hour, anthranilic acid (161 mg, 1.17 mmol, 1.3 equiv) and pyridinium p-toluene sulfonate (543 mg, 2.16 mmol, 2.4 equiv) were added and the mixture was refluxed under nitrogen. After 18 hours the mixture was cooled to room temperature before being filtered through Celite and evaporated under reduced pressure. Purification by SPE (C18, 20 g) eluting 3-95% MeCN: water, afforded the title compound as a white solid (80 mg, 33%); δ_{H} (400 MHz, d6-DMSO) 2.83 (2H, t, J=7.5 Hz), 3.12 (2H, t, J=7.5 Hz), 7.13 (1H, app t, J=7.5 Hz), 7.43-7.49 (3H, m), 7.57 (1H, app t, J=8.0 Hz), 7.76 (1H, br s), 7.82-7.87 (3H, m), 7.96 (1H, dd, J=8.0 and 1.6 Hz), 8.48 (1H, d, J=8.5 Hz), 11.18 (1H, s), 13.61 (1H, br s); m/z 320.2 [MH+].

### Example 25

### 2-{[3-(1,2,3,4-tetrahydro-1-naphthalenyl)propanoyl]amino}benzoic acid

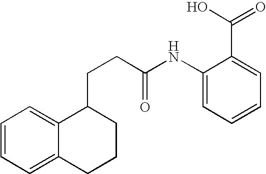

Prepared as for Example 24 using 3-(1,2,3,4-tetrahydro-1-naphthalenyl)propanoic acid (100 mg, 0.49 mmol, 1 equiv) and anthranilic acid (88 mg, 0.64 mmol, 1.3 equiv). Purification by SPE (C18, 5 g) eluting 3-95% MeCN: water, afforded the title compound as a tan oil (45 mg, 28%); δ_{H} (400 MHz, DMSO-d⁶) 1.60-1.72 (2H, m), 1.73-1.89 (3H, m), 2.00-2.10 (1H, m), 2.40-2.52 (2H, m), 2.65-2.85 (3H, m), 7.00-7.18 (4H, m), 7.21 (1H, d, J=7.5 Hz), 7.57 (1H, t, J=8.0 Hz), 7.97 (1H, d, J=8.0 Hz), 8.48 (1H, d, J=8.0 Hz), 11.18 (1H, s), 13.60 (1H, v br s); m/z 324.2 [MH⁺].

### B. Example Compounds Synthesised Using Method B

A generalized method for carrying out Method B is as follows:

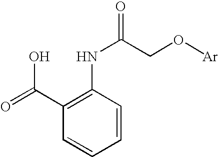

Polystyrene-supported 1,5,7-triazabicyclo[4.4.0]dec-5-ene [Fluka AG, crosslinked with 2% 1,4-divinylbenzene, loading 2.6 mmol/g] is treated with a solution of Ar—OH in acetonitrile. After 1 hour the mixture is treated with a solution of methyl 2-[(chloroacetyl)amino]benzoate (Journal of Heterocyclic Chemistry 1989, 26(6), 1807-1810) in acetonitrile and then heated at around 45° C. for around 18 hours. The cooled mixture is filtered and the residue washed with further acetonitrile. The filtrate and washings are combined, evaporated to dryness and the residue treated with a solution of lithium hydroxide in a mixture of methanol, water and THF. The mixture is stirred and heated to around 45° C. for about 2 hours then stirred at ambient temperature for around 18 hours. The mixture is acidified to about pH4 by the addition of 2M aqueous hydrochloric acid and the precipitated product filtered and dried to afford the title compound.

The following compounds example 26-34 were also prepared using method A

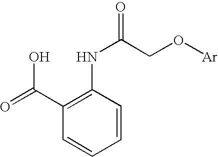

| Example No: | Compound: Ar = | Yield | m/z |
|---|---|---|---|
| 26 | 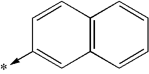 | 2.8mg(5.8%) | 322[MH⁺] |
| 27 | 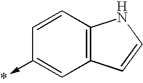 | 3.6mg(7.7%) | 311[MH⁺] |
| 28 | 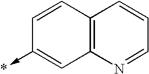 | 3.2mg(6.6%) | 323[MH⁺] |
| 29 | 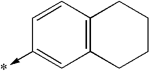 | 2.9mg(5.9%) | 326[MH⁺] |
| 30 | 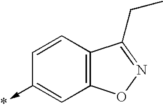 | 21.6mg(42.2%) | 341[MH⁺] |
| 31 | 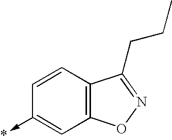 | 14.4mg(27%) | 355[MH⁺] |
| 32 | 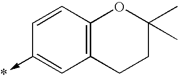 | 20.5mg(38.4%) | 356[MH⁺] |
| 33 | 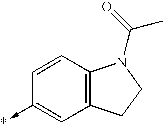 | 1.6mg(3%) | 355[MH⁺] |

### Example 26

### 2-{[(2-naphthalenyloxy)acetyl]amino}benzoic acid

NMR δH (400 MHz, d6-DMSO) 4.87(s, 2H), 7.20(t, 1H, J=7.8 Hz), 7.35-7.41(m, 2H), 7.45-7.51(m, 2H), 7.64(dd, 1H, J=1.5, 7.0 Hz), 7.83(d, 1H, J=8.3 Hz), 7.87(d, 1H, J=8.0 Hz), 7.92(d, 1H, J=8.8 Hz), 8.03(dd, 1H, J=1.5, 7.8 Hz), 8.73(d, 1H, J=8.3 Hz), 12.31 (s, 1H), one exchangeable proton not observed to δH 13.

### Example 27

### 2-{[(1H-indol-5-yloxy)acetyl]amino}benzoic acid

NMR δH (400 MHz, d6-DMSO) 4.69 (s, 2H), 6.33-6.38 (m, 1H), 6.94 (dd, 1H, J=2.5, 8.6 Hz), 7.17-7.20 (m, 3H), 7.31-7.34 (m, 2H), (dt, 1H, J=1.5, 7.3 Hz), 8.02 (dd, 1H, J=1.5 and 7.8 Hz), 8.73 (d, 1H, J=8.1 Hz), 11.00 (s, 1H), 12.28 (s, 1H).

### Example 28

### 2-{[(7-quinolinyloxy)acetyl]amino}benzoic acid

NMR δH (400 MHz, d6-DMSO) 4.93 (s, 2H), 7.18 (t, 1H, J=7.1 Hz), 7.40-7.51 (m, 3H), 7.62 (dt, 1H, J=1.5, 8.6 Hz), 7.98 (d, 1H, J=8.8 Hz), 8.03 (dd, 1H, J=1.5, 7.8 Hz), 8.31 (dd, 1H, J=1.5, 8.3 Hz), 8.71 (d, 1H, J=8.1 Hz), 8.84 (dd, 1H, J=1.7, 4.3 Hz), 12.46 (br s, 1H), one exchangeable proton not observed to δH 13.

### Example 29

### 2-{[(5,6,7,8-tetrahydro-2-naphthalenyloxy)acetyl]amino}benzoic acid

NMR δH (400 MHz, d6-DMSO) 1.75-1.96 (m, 4H), 2.69-2.87 (m, 2H), 2.88-2.96 (m, 2H), 4.67 (s, 2H), 6.92-6.99 (m, 2H), 7.09-7.16 (m, 2H), 7.41 (t, 1H, J=8.4 Hz), 8.13 (d, 1H, J=8.4 Hz), 8.68 (d, 1H, J=8.4 Hz), both exchangeable protons not observed to δH 13.

### Example 30

### 2-({[(3-ethyl-1,2-benzisoxazol-6-yl)oxy]acetyl}amino)benzoic acid

NMR δH (400 MHz, d6-DMSO) 1.33 (t, 3H, J=7.5 Hz), 2.96 (q, 2H, J=7.5 Hz), 4.82 (s, 2H), 7.11 (t, 1H, J=7.5 Hz), 7.17 (dd, 1H, J=1.5, 8.5 Hz), 7.37 (d, 1H, J=1.8 Hz), 7.49 (t, 1H, J=6.5 Hz), 7.82 (d, 1H, J=8.8 Hz), 8.01 (d, 1H, J=7.8 Hz), 8.63 (d, 1H, J=8.5 Hz), both exchangeable protons not observed to δH 13.

### Example 31

### 2-({[(3-propyl-1,2-benzisoxazol-6-yl)oxy]acetyl}amino)benzoic acid

NMR δH (400 MHz, d6-DMSO) 0.96 (t, 3H, J=7.5 Hz), 1.78 (m, 2H), 2.92 (t, 2H, J=7.5 Hz), 4.86 (s, 2H), 7.15-7.19 (m, 2H), 7.39 (d, 1H, J=2.0 Hz), 7.59 (t, 1H, J=8.0 Hz), 7.84 (d, 1H, J=8.5 Hz), 8.03 (d, 1H, J=7.8 Hz), 8.68 (d, 1H, J=8.3 Hz), 12.41-12.83 (br s,1H), one exchangeable proton not observed to δH 13.

### Example 32

### 2-({[(2,2-dimethyl-3,4-dihydro-2H-chromen-6-yl)oxy]acetyl}amino)benzoic acid

NMR δH (400 MHz, d6-DMSO) 1.24 (s, 6H), 1.72 (t, 2H, J=6.5 Hz), 2.71 (t, 2H, J=6.5 Hz), 4.59 (s, 2H), 6.65-6.67 (m, 1H), 6.81-6.84 (m, 2H), 7.14 (t, 1H, J=7.5 Hz), 7.55 (t, 1H, J=7.3 Hz), 8.02 (dd, 1H, J=1.3, 7.8 Hz), 8.67 (d, 1H, J=8.3 Hz), 12.65-12.82 (br s, 1H), one exchangeable proton not observed to δH 13.

### Example 33

### 2-({[(1-acetyl-2,3-dihydro-1H-indol-5-yl)oxy]acetyl}amino)benzoic acid

NMR δH (400 MHz, d6-DMSO), 2.19(s, 3H), 3.19(t, 2H, J=8.3 Hz), 4.10(t, 2H, J=8.3 Hz), 4.59 (2, 2H), 6.90(d, 1H, J=9.1 Hz), 7.04-7.11(m, 2H), 7.37(t, 1H, J=7.6 Hz), 7.89-8.07(m, 2H), 8.57(d,1H, J=8.3 Hz), both exchangeable protons not observed to δH 13.

All publications, including but not limited to patents and patent applications, cited in this specification are herein incorporated by reference as if each individual publication were specifically and individually indicated to be incorporated by reference herein as though fully set forth.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation the following claims:

1. A compound selected from a compound of Formula (I): 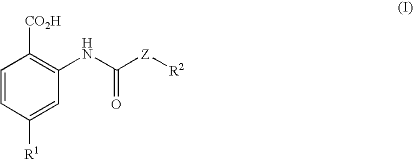
or a salt, solvate or physiologically functional derivative thereof, wherein:
R¹ is hydrogen, halogen or C₁-C₃alkyl;
R² is a 9 or 10-member saturated, partially saturated or unsaturated bi-cyclic ring system optionally including from 1 to 3 heteroatoms independently selected from S, O and N;
Z is —(CH₂)ₙ—, —CH═CH—(CH₂)ₘ—, —(CH₂)ₚNHC(O)—, —(CH₂)ₚNHC(O)NH—, —(CH₂)ₚNHC(O)O—, —(CH₂)ₚSO₂NR³—, —(CH₂)ₚNR³SO₂—, —(CH₂)ₚO— or —O—;
n is 2, 3 or 4;
m is 0, 1 or 2;
p is 1 or 2; and
R³ represents hydrogen or C₁-C₄alkyl, with the proviso that when R¹ is H, Z is —(CH₂)ₙ— and n is 2 or 3, R² is other than indol-3-yl.

2. A compound according to claim 1 wherein R¹ is hydrogen, fluorine or methyl.

3. A compound according to claim 2 wherein R¹ is hydrogen.

4. A compound according to claim 1 wherein Z is —(CH₂)ₚO— or —(CH₂)ₙ—.

5. A compound according to claim 4 wherein Z is —(CH₂)ₙ— and n is 2, 3 or 4.

6. A compound according to claim 5 wherein Z is —(CH₂)ₙ— and n is 2.

7. A compound according to claim 4 wherein Z is —(CH₂)ₚO— and n is 1.

8. A compound according to claim 1 wherein R² is a 10-member bi-cyclic ring system.

9. A compound according to claim 8 wherein R² is naphthyl.

10. A compound according to claim 8 wherein R² is a 10-member ring system having 1 or 2 heteroatoms.

11. A compound according to claim 10 wherein R² contains 1 or 2 nitrogen heteroatoms.

12. A compound according to claim 8 wherein R² is selected from the group consisting of:

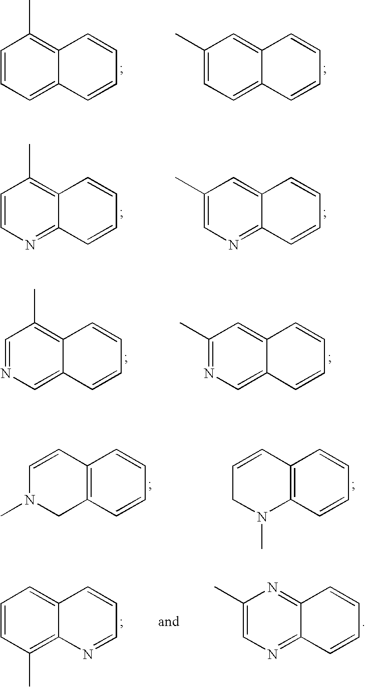

13. A compound according to claim 8 wherein R² is substituted with one or more groups which are C₁-C₂alkyl, —C(O)Me, ═O or C₁-C₃alkoxy.

14. A compound according to claim 13 wherein R² is substituted with one or more groups which are methyl or methoxy.

15. A compound according to claim 1 wherein R² is a 9-member ring system selected from the group consisting of fused aryl-cycloalkyl, fused aryl and fused heteroaryl systems.

16. A compound according to claim 15 wherein R² contains 1 to 3 heteroatoms which are S, O or N.

17. A compound according to claim 15 wherein R² is selected from the group consisting of: 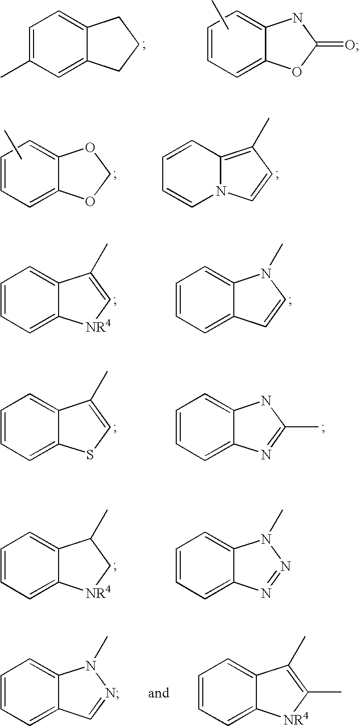
wherein R⁴ is hydrogen, methyl, CO₂H or CO₂Me.

18. A compound according to claim 17 wherein R² is substituted with one or more groups which are C₁-C₃alkyl —C(O)Me, ═O, C₁-C₃alkoxy, CO₂H and CO₂Me.

19. A compound according to claim 18 wherein R² is substituted with methyl or methoxy.

20-26. (canceled)

27. A method for the treatment of a human or animal subject having a condition where under-activation of the HM74A receptor contributes to the condition or where activation of the receptor will be beneficial, which method comprises administering to said human or animal subject an effective amount of a compound of Formula (Ia): 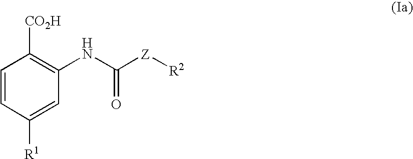
or a pharmaceutically acceptable salt, solvate or physiologically functional derivative thereof, wherein:
R¹ is hydrogen, halogen or C₁-C₃alkyl;
R² is a 9 or 10-member saturated, partially saturated or unsaturated bi-cyclic ring system optionally including from 1 to 3 heteroatoms independently selected from S, O and N;
Z is —(CH₂)ₙ—, —CH═CH—(CH₂)ₘ—, —(CH₂)ₚNHC(O)—, —(CH₂)ₚNHC(O)NH—, —(CH₂)ₚNHC(O)O—, —(CH₂)ₚSO₂NR³—, —(CH₂)ₚNR³SO₂—; or —O—;
n is 2, 3 or 4;
m is 0, 1 or 2;
p is 1 or 2; and
R³ is hydrogen or C₁-C₄alkyl.

28. A method for the treatment of a human or animal subject having a disorder of lipid metabolism or having an inflammatory disease, which method comprises administering to said human or animal subject an effective amount of a compound of Formula (Ia): 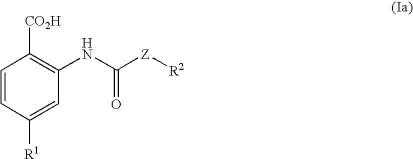
or a pharmaceutically acceptable salt, solvate or physiologically functional derivative thereof, wherein:
R¹ is hydrogen, halogen or C₁-C₃alkyl;
R² is a 9 or 10-member saturated, partially saturated or unsaturated bi-cyclic ring system optionally including from 1 to 3 heteroatoms independently selected from S, O and N;
Z is —(CH₂)ₙ—, —CH═CH—(CH₂)ₘ—, —(CH₂)ₚNHC(O)—, —(CH₂)ₚNHC(O)NH—, —(CH₂)ₚNHC(O)O—, —(CH₂)ₚSO₂NR³—, —(CH₂)ₚNR³SO₂—; or —O—;
n is 2, 3 or 4;
m is 0, 1 or 2;
p is 1 or 2; and
R³ is hydrogen or C₁-C₄alkyl.

29. A pharmaceutical formulation comprising a compound according to claim 1 in admixture with one or more physiologically acceptable diluents, excipients or carriers.

30. A combination for administration together or separately, sequentially or simultaneously in separate or combined pharmaceutical formulations, said combination comprising a compound according to claim 1 together with another therapeutically active agent.

31. A pharmaceutical formulation comprising a compound according to claim 1, a further active ingredient selected from the group consisting of statins, fibrates, bile-acid binding resins and nicotinic acid and one or more physiologically acceptable diluents, excipients or carriers.

32. A process for the preparation of a compound according to claim 1, the process comprising the steps of:
i. alkylation of an aromatic alcohol with methyl 2-[(chloroacetyl)amino]benzoate;
ii hydrolysis of methyl ester using lithium hydroxide; and
iii where desired or necessary converting a resultant free base or acid compound of Formula (I) into a physiologically acceptable salt or free base or converting one salt into another physiologically acceptable salt.

33. A process for the preparation of a compound according to claim 1, the process comprising the steps of:
i. formation of an amide between the amine group of anthranilic acid (2-amino-bezoic acid) and an activated acyl transfer reagent derived from a carboxylic acid; and
ii where desired or necessary converting a resultant free base or acid compound of Formula (I) into a physiologically acceptable salt or free base or converting one salt into another physiologically acceptable salt.

34. A method according to claim 28 wherein the disorder of lipid metabolism is dislipidaemia or hyperlipoproteinaemia.
